(19) United States
(12) Patent Application Publication
(10) Pub. No.: US 2022/0008854 A1
(43) Pub. Date: Jan. 13, 2022

(54) ANTIVIRAL FILTER MEDIUM

(71) Applicant: Carl Freudenberg KG, Weinheim (DE)

(72) Inventors: Uwe Haefner, Kehl (DE); Heiko Schacht, Weinheim (DE); Ilker Uenen, Osthofen (DE); Sascha Blauth, Rodenbach (DE)

(21) Appl. No.: 17/367,436

(22) Filed: Jul. 5, 2021

(30) Foreign Application Priority Data
Jul. 9, 2020 (DE) 10 2020 118 182.5

(51) Int. Cl. B01D 46/00; A61L 9/16; B01D 39/00

(52) U.S. Cl. CPC B01D 46/0028; B01D 46/0036; A61L 9/16; B01D 39/00; B01D 2279/40; B01D 2239/0478; B01D 2239/0618; B01D 2257/91; A61L 2209/14; B01D 2239/0442

(57) ABSTRACT

A method includes: using a planar substrate having at least one acid-functionalized layer including at least one first acid having a pks1 value of 0 to 7 and at least one different second acid or derivative thereof selected from a group consisting of C₈ to C₁₈ fatty acids, esters, amides, and mixtures thereof, as a filter medium for depleting viruses from air and other gases.

### CROSS-REFERENCE TO PRIOR APPLICATION

Priority is claimed to German Patent Application No. DE 10 2020 118 182.5, filed on Jul. 9, 2020, the entire disclosure of which is hereby incorporated by reference herein.

### FIELD

The present invention relates to the use of a planar substrate as a filter medium for depleting viral pathogens from the air and other gases and to a method for depleting viral pathogens from the air and other gases.

### BACKGROUND

Viruses are infectious organic structures that spread as virions outside of cells (extracellularly) through transmission, but can reproduce as viruses only within a suitable host cell (intracellularly). They themselves do not consist of one or more cells. All viruses contain the program for their reproduction and spread (some viruses also contain further auxiliary components), but have neither an independent replication nor a separate metabolism and therefore depend on the metabolism of a host cell. The viruses couple to surface molecules of the host cells and introduce their genetic material into them. The genetic material penetrates into the nucleus and changes the cell's own DNA. There is, inter alia, massive replication of the virus body (genome and proteins) in the affected cell by the cell organelles present.

A virus particle outside of cells is referred to as virion. Virions are particles containing nucleic acids—either deoxyribonucleic acids (DNA) or ribonucleic acids (RNA)—and mostly having an enclosing protein capsule (capsid). However, a capsule is absent, for example, in influenza virus, which instead has a ribonucleoprotein. Some virions additionally have a biomembrane envelope, the lipid bilayer of which is interspersed with viral membrane proteins. This is referred to as viral envelope. Viruses which in addition to the capsid temporarily have a viral envelope up to the beginning of the replication phase, are referred to as enveloped, whereas viruses without such an envelope are referred to as non-enveloped.

The diameter of virions is about 15 nm (e. g., Circoviridae) to 440 nm (Megavirus chilensis). Virions are much smaller than bacteria, but somewhat bigger than viroids which have neither a capsid nor a viral envelope.

Coronaviruses (CoV) are “enveloped viruses” of the Coronavirinae subfamily in the family of Coronaviridae. They can cause diseases from colds to more severe diseases such as the Middle East respiratory syndrome (MERS-CoV) or the severe acute respiratory syndrome (SARS-CoV). The novel SARS-CoV-2 coronavirus is a new strain which in the past has not been found in humans.

Filter materials for purifying air, in particular for cleaning the air of dusts, suspended matter or allergens, such as pollen and mites, are known from the prior art.

DE 10 2016 212 056 describes a filter medium based on a cationic ion exchanger and an anti-pathogenic substance, such as polyphenols. The ion exchanger forms an acidic environment with water; in connection with the anti-pathogenic substance, this combination is harmful to some microorganisms. It is expressly mentioned that the acidic protons reduce or stop the biological activity of bacteria, germs, fungi and algae (and not of viruses). This is intended to solve the problem of these special microorganisms reproducing in the filter material, which problem occurs in filter media, especially in vehicle air conditioning systems. This differs from viral pathogens that are biologically active and can reproduce only in the presence of host cells. Thus, said document has a different underlying object than the present invention which is intended to remove viral pathogens from the air and other gases.

EP 3 162 425 describes a filter material for removing allergens from the air. The filter material comprises an acid-functionalized layer comprising a fruit acid and a fatty acid.

For the field of simple respiratory applications, e.g. in filter masks, it is known to clean air from viral pathogens. Also known from the prior art are individual filter materials for cleaning the air in stationary and mobile air treatment systems (e.g. filter systems for room air purification or for vehicle air conditioning).

DE 10 2013 021 071 A1 describes a filter medium, in particular for filtering air for the interior of motor vehicles, comprising an antimicrobial and an anti-allergenic substance. The antimicrobial substance is selected from a plurality of different compounds, such as metals and metal compounds, etc. The filter medium is intended to be capable of killing microorganisms, in particular fungi and fungal spores, and at the same time effectively preventing bacteria, fungi and other microorganisms from growing on the filter medium. Said document, too, does not mention employing a specially equipped filter medium to bind and inactivate viral pathogens that are biologically active and can reproduce only in host cells. Thus, said document also has a different object than the present invention.

U.S. Pat. No. 5,888,527 describes an antifugal, antibacterial and antiviral filter comprising a dust-collecting filter nonwoven with a tea extract finish. This filter is to be suitable for binding and inactivating viruses and preventing new spreading.

WO 2014/019660 A1 describes an anti-allergenic filter for the ventilation system for the interior of motor vehicles. The filter substrate is equipped with a polyphenol from the class of tannins as an anti-allergenic compound. In addition, the filter substrate may contain zinc oxide as an antibacterial agent.

There is currently an immense need for filter media that are capable of effectively removing viruses from the air or other gases. This applies in particular to coronaviruses such as SARS-CoV-2 or MERS-CoV, and to influenza viruses such as the influenza virus A variant H1N1.

### SUMMARY

In an embodiment, the present invention provides a method, comprising: using a planar substrate comprising at least one acid-functionalized layer comprising at least one first acid having a pks1 value of 0 to 7 and at least one different second acid or derivative thereof selected from a group consisting of C₈ to C₁₈ fatty acids, esters, amides, and mixtures thereof, as a filter medium for depleting viruses from air and other gases.

### DETAILED DESCRIPTION

In an embodiment, the present invention provides a filter medium which can be used for depleting viruses from air and gases. In particular, the viruses are to be not only deposited on and/or in the filter medium, but also inactivated. On the one hand, this has the advantage that any viral material still contained in the air that exits the filter medium is inactivated and no longer pathogenic. In addition, the loaded filter medium also has essentially no more pathogenic material.

In an embodiment, the present invention provides using using a planar substrate having at least one specific acid-functionalized layer as a filter medium and a method according to the invention for purifying air.

The use according to the invention has the particular advantage of an antiviral action of the filter media against various viral strains, e.g. H1N1 and HCoV229E.

A first subject of the invention is to use a planar substrate comprising or consisting of at least one acid-functionalized layer comprising a first acid having a pks1 value of 0 to 7 and a different second acid or a derivative thereof selected from C₈ to C₁₈ fatty acids, esters, amides and mixtures thereof, as filter medium for depleting viruses from the air and other gases.

Another subject of the invention is a method for depleting viral pathogens from the air or other gases, comprising the steps of
i) introducing virus-enriched air or gas into a filter device comprising at least one planar substrate, as previously and hereinafter defined, as a filter medium,
ii) directing the air or the gas through the filter medium or contacting the air or the gas with the filter medium to obtain air depleted of viruses or gas depleted of viruses,
iii) discharging the air depleted of viruses or the gas depleted of viruses from the filter device.

Another subject is a planar substrate, as previously and hereinafter defined, for depleting viruses from the air or other gases.

The expression “depleting viruses” as used in the invention also means inactivating them. Air or other gases containing pathogenic viruses are passed through a planar substrate comprising at least one acid-functionalized layer, as defined hereinabove and hereinafter, and serving as a filter medium. At least some of the viruses contained in the air or the gas are bound by the filter medium and the virus concentration is thus reduced by physical deposition. In addition, at least some of the viruses contained in the air or the gas are inactivated by contact with the acid-functionalized layer (chemical deactivation) so that they are no longer pathogen-effective. Even if said amount of inactivated viruses is not retained completely in the filter medium, the concentration of pathogenic viruses in the air or the gas is reduced also by the inactivation. The use according to the invention makes it possible to obtain air or gases which are free of viral pathogens or contain them in a concentration that is so low that an infection of humans having contact with, especially inhaling, this air or gas or spending a longer period of time in a room containing this air or gas is excluded. Pathogenic viruses are substantially completely removed by the use according to the invention. Preferably, by contacting the virus-loaded air or gas with the planar substrate comprising or consisting of at least one acid-functionalized layer, a viral pathogen reduction factor of preferably >3.0 log stages, particularly preferably >5.0 log stages, is achieved. This reduction in pathogenicity is due to the deactivation of viruses by the acid-functionalized layer. The antiviral properties can be determined according to ISO 18184:2019-06 for determining the antiviral activity of textile products or comparable methods.

The use according to the invention of a planar substrate, which comprises or consists of at least one acid-functionalized layer, as a filter medium is suitable in general for depleting viruses from a gas or a mixture of two or more different gases. A preferred gas mixture is air. The use as a filter medium for depleting viruses from a breathable gas mixture is also preferred. Breathable gas mixtures contain oxygen and at least one inert gas which is not involved in the metabolic processes and serves to dilute the oxygen. Suitable inert gases are nitrogen, helium, neon and hydrogen.

According to the invention, it has been found that the combination of an acid having a pks1 value of 0 to 7 and a C₈ to C₁₈ fatty acid makes it possible to equip filter media with a high capacity for deactivating viruses. This was surprising since it was assumed that fatty acids would block the activity of the antiviral acids due to their oily nature. Indeed, in practical trials, a reduction in the deactivation activity of these acids applied to filter media has been found, but to a much lesser extent than assumed. Without wanting to commit to any mechanism according to the invention, it is assumed that the two acid classes act synergistically in that the fatty acids as oily substances improve the deposition and fixation of the viruses on the filter medium, thereby at least partially compensating for the blocking of the antiviral acids.

Viruses in terms of the invention are enveloped and non-enveloped viruses.

Enveloped viruses are preferably selected from among Coronaviridae, Orthomyxoviridae and Pneumoviridae.

Coronaviridae are preferably selected from among coronavirus 229E (HCoV-229E), coronavirus NL63 (HCoV-NL63), coronavirus 0C43 (HCoV-OC43), coronavirus HKU1 (HCoV-HKU1), MERS-CoV (Middle East respiratory syndrome-related coronavirus) and SARS-associated coronavirus (SARS-CoV)—with subtype SARS-CoV-2, in particular COVID-19.

Orthomyxoviridae are preferably selected from among Influenza virus A, Influenza virus B, Influenza virus C and Influenza virus D.

Influenza virus A is specifically Influenza virus A variant H1N1, Influenza virus A variant H3N2, Influenza virus A variant H5N1.

Influenza virus B is specifically Influenza virus B/Victoria Line and Influenza virus B/Yamagata Line.

Pneumoviridae are specifically respiratory syncytial virus (HRSV) (type A, B) and metapneumovirus (HMPV) (type A1 to 2, B1 to 2).

Non-enveloped viruses are specifically selected from among Picornaviridae.

Picornaviridae are specifically selected from among Coxsackievirus A/B, Coxsackievirus B1 (CVB-1), echovirus, enterovirus and rhinovirus.

Rhinoviruses are specifically rhinoviruses-1 A (HRV-1 A), 1 B to 100.

A preferred embodiment is the use of the filter medium as defined above and below for depleting Coronaviridae and Orthomyxoviridae from the air and gases, in particular for depleting SARS-associated coronavirus, the Middle East respiratory syndrome-related coronavirus (MERS-CoV) and Influenza virus A from the air and gases, specifically for depleting SARS-CoV-2, MERS-CoV and Influenza virus A variant H1N1.

The filter medium used according to the invention comprises at least one acid-functionalized layer comprising a first acid having a pks1 value of 0 to 7 and a second acid different therefrom and selected from among C₈ to C₁₈ fatty acids, esters, amides and mixtures thereof.

The pKs value (acid constant) is a measure of the strength of an acid. Acidity is all the more, the lower its pKs value.

The pKs values can be determined via acid base titrations and the determination of the pH at the half equivalent point. Here, the acid and its corresponding base are present in the same concentration. At this point, the following follows from the Henderson Hasselbalch equation: pH=pKs.

The first acid preferably has a pks1 value of 1.0 to 5.0, in particular of 2.0 to 4.0 and in particular of 2.5 to 4.0.

In a preferred embodiment, the first acid comprises a fruit acid.

Fruit acids are organic hydroxycarboxylic acids, dicarboxylic acids and tricarboxylic acids, wherein some fruit acids can be assigned to both the hydroxycarboxylic acids and dicarboxylic acids or tricarboxylic acids.

Suitable hydroxy acids are selected from among fumaric acid, gluconic acid, glycolic acid, mandelic acid, lactic acid, salicylic acid, α-hydroxycaprylic acid, and mixtures thereof

Suitable dicarboxylic acids are selected from among malic acid, oxalic acid, tartaric acid and mixtures thereof.

A preferred tricarboxylic acid is citric acid.

In another embodiment, the first acid is selected from among malic acid, citric acid, fumaric acid, gluconic acid, glycolic acid, mandelic acid, lactic acid, oxalic acid, salicylic acid, a-hydroxycaprylic acid, tartaric acid, and mixtures thereof. More preferably, the first acid comprises or consists of citric acid.

The second acid of the acid-functionalized layer is an acid different from the first acid and is selected from among C₈ to C₁₈ fatty acids, esters, amides or mixtures thereof.

Suitable fatty acids are saturated or monounsaturated or polyunsaturated aliphatic monocarboxylic acids with mostly unbranched carbon chain. These are preferably C₈ to C₁₈ fatty acids having predominantly linear alkyl radicals or predominantly linear alkenyl radicals, as are also contained in natural or synthetic fatty acids which may be saturated or which may be mono-, di-, tri-, tetra-, penta-, or hexa-unsaturated. Fatty acids according to the invention, selected from among C₈ to C₁₆ fatty acids and mixtures thereof have proven to be particularly suitable. In a particular embodiment, the second acid is selected from among saturated linear C₁₂ to C₁₄ fatty acids and mixtures thereof. In another particular embodiment, the second acid is selected from among saturated linear C₈, C₁₀ and C₁₂ fatty acids and mixtures thereof The fatty acids preferably have an unbranched carbon chain.

The use of caprylic acid, pelargonic acid, capric acid, undecanoic acid, lauric acid, tridecanoic acid, myristic acid, pentadecanoic acid, palmitic acid and/or mixtures thereof has proven to be particularly effective as a second acid.

Also suitable are fatty acid derivatives, in particular fatty acids which contain hydroxy groups as functional radicals, as well as fatty acid esters, fatty acid amides, in particular oleic acid amides and stearic acid amides and/or mixtures thereof.

The molecules of the most common fatty acids have 16 or 18 carbon atoms. They are hence particularly inexpensive. In addition, the sodium and potassium salts of these fatty acids have the advantage of acting as surfactant.

According to the invention, less water-soluble to virtually insoluble C₈ to C₁₈ fatty acids are particularly suitable as the second acid.

Lauric acid is particularly preferably used. Lauric acid is a very mild antimicrobial substance and its application therefore is not subject to strong regulations. Nevertheless, lauric acid exhibits a very good antiviral effect in the filter medium according to the invention.

The combination of lauric acid and citric acid is particularly preferred according to the invention. In practical trials it has been confirmed that this combination can provide a filter medium with an outstanding antiviral effect for a long period of time, preferably over the entire duration of filter use. Moreover, both compounds exhibit good environmental compatibility and, during their processing, do not have extraordinary safety at work requirements.

The ratio of the first acid to the second acid in the acid-functionalized layer can be adjusted as a function of the desired performance of the filter medium. Weight ratios in the range of from 10,000:1 to 1:1, preferably of from 1000:1 to 2:1, more preferably 100:1 to 5:1 have proven particularly favorable.

The amount of the first acid and second acid in the acid-functionalized layer can also be adjusted as a function of the desired performance of the filter medium. The amount of the first acid in the acid-functionalized layer is preferably 0.1 wt % to 30 wt %, preferably from 2 wt % to 24 wt %, even more preferably from 6 wt % to 18 wt %, more preferably from 7 wt % to 15 wt % and in particular from 8 wt % to 12 wt % based in each case on the total weight of the acid-functionalized layer. The amount of the second acid in the acid-functionalized layer is preferably less than 10 wt %, preferably from 0.01 wt % to 5 wt %, even more preferably from 0.02 wt % to 1 wt %, more preferably from 0.04 wt % to 0.6 wt % and in particular from 0.08 wt % to 0.12 wt % based in each case on the total weight of the acid-functionalized layer. The total weight of the acid-functionalized layer here comprises first acid, second acid, carrier material and, if present, binders, wetting agents and further additives. It has been found that at a greater concentration of fatty acid, the antiviral activity of the first acid is reduced too much.

In practical experiments, it has also been found that a planar substrate used as a filter medium according to the invention exhibits excellent deactivation of viruses combined with a biocidal effect compared to other microorganisms even with a comparatively low amount of fatty acid.

The planar substrate used according to the invention comprises or consists of at least one acid-functionalized layer. To produce the acid-functionalized layer, a carrier material can be impregnated and/or coated with at least one first acid and with at least one second acid. The layer to be functionalized can be provided with the first and the second acid in various ways known to the person skilled in the art, such as by means of impregnation and/or coating, for example, panning, padding, spraying and/or dipping. The layer to be functionalized can thus be impregnated and/or coated in a simple manner with a solution and/or suspension containing the first and second acid. Likewise conceivable is the impregnation and/or coating of the layer with a mixture of binder, for example a thermoplastic binder containing the first and second acid.

In a further embodiment, the first acid and/or second acid is used in the form of a pourable or free-flowing solid to produce the acid-functionalized layer. In this case, the first acid and/or the second acid can be sprinkled dry into the carrier material. The thus resulting filter media have the advantage of being simple to produce since the pourable or free-flowing solid is simple to handle.

In particular, the free-flowing first and/or second acid is a granulate. Suitable granules are in the form of a powder, spheres, granules, particles, dust or mixtures thereof. The granules preferably have a diameter of 200 to 700 μm. Said diameter is also referred to as grain size. When using granules with a grain size of >700 μm, a uniform distribution of the acids over the surfaces of the filter medium is generally achieved. In particular, the acids are present in a concentration of 2-250 g/m², more preferably 20-25 g/m².

In a further embodiment, the first acid is used in the form of a pourable or free-flowing solid for producing the acid-functionalized layer and the second acid is applied to the acid-functionalized layer by means of impregnation and/or coating.

In a preferred embodiment, the acid-functionalized layer comprises
at least one first acid having a pks1 value of 0 to 7,
at least one second acid different from the first acid or a derivative thereof selected from among C₈ to C₁₈ fatty acids, esters, amides and mixtures thereof,
at least one carrier material,
optionally at least one binder,
optionally at least one wetting agent,
optionally at least one further additive, for example selected from among compounds eliminating allergens, fungicides, etc.

Nonwovens, wovens, warp-knitted fabrics and/or papers can preferably be used as carrier materials for the acid-functionalized layer. A particularly preferred embodiment according to the invention thus comprises the embodiment of the acid-functionalized layer as impregnated and/or coated nonwoven, as impregnated and/or coated woven, warp-knitted fabric and/or paper. The use of a nonwoven is particularly preferred according to the invention.

The planar substrate used according to the invention comprises or consists of at least one acid-functionalized layer. The planar substrate may have a single layer or multiple layers. In a first embodiment, the planar substrate consists of at least one acid-functionalized layer as described above. In a further embodiment, the planar substrate consists of at least one acid-functionalized layer, as described above, and at least one layer different therefrom. The at least one layer which is different from the acid-functionalized layer can be modified neither with one of the aforementioned first acids nor with one of the aforementioned second acids or only with one of the aforementioned first acids or only with one of the aforementioned second acids. In a suitable embodiment, the planar substrate is present as a two-layer or multilayer sheet. In that case, the sheet has, for example, at least one acid-functionalized layer and at least one further layer which is selected, for example, from among nonwovens, rovings, wovens, knitted fabrics, warp-knitted fabrics, papers and combinations thereof.

For the purposes of the invention, the term nonwoven as used herein relates to a fabric consisting of fibers of limited length, continuous fibers (filaments) or cut yarns of any type and of any origin which have in some way been joined together to form a fibrous layer or a fibrous web and have in some way been connected to one another; excluded therefrom is the interlacing or interweaving of yarns, as occurs during weaving, warp-knitting, knitting, lace-making, braiding and the production of tufted products. Nonwovens do not include films and papers, for example.

In a particularly preferred embodiment of the invention, the layer to be functionalized is treated with a surfactant as wetting agent, preferably one or more nonionic surfactants as wetting agents, more preferably with ethoxylated sorbitan fatty acid esters (polysorbates) before and/or simultaneously with the application of the second acid. Particular preference is given to polysorbates which, based on Regulation (EC) No. 1333/2008 of the European Parliament and of the Council of Dec. 16, 2008, are approved as food additive in the European Union, such as E 432, E 434, E 435 and E 436.

In particular, the planar substrate used in accordance with the invention is free of polyoxyethylene (20) sorbitan monooleate (polysorbate 80, E433).

The advantage of using wetting agents is that the first and/or second acid can be fixed particularly well on the layer to be functionalized. This enables good immobilization and deactivation of the viruses. With regard to the use of odor-intensive active substances, the surfactant offers the additional advantage that the immobilization of these substances can also reduce odor release.

The planar substrate can furthermore also comprise further allergen-eliminating compounds, such as polyphenols, in particular flavonoids, phenolic acids, polyhydroxyphenols, anthocyanins, procyanidins, benzoic acid and stilbene derivatives, preferably of natural origin, such as, for example, the secondary plants materials present in pomegranates, ginkgos or grape seed flour, and/or mixtures thereof. These compounds are preferably present in an amount of 2% to 20%, based in each case on the total weight of the filter medium.

The planar substrate may also contain fungicidal agents. For this purpose, the acid-functionalized layer can be treated with a fungicidal substance before and/or simultaneously with the application of the fatty acid, preferably with triazoles such as, in particular, 2-octyl-2H-isothiazole-3-on and/or metals and their compounds, for example zinc pyrethiones.

The planar substrate according to the invention is perfectly suitable for use as a filter medium for depleting viruses from the air of buildings, building parts and mobile facilities. This includes, on the one hand, the air between the building, building part or mobile facility exchanged with the outside world, especially the supplied fresh air (outside air) and the discharged exhaust air (outgoing air). To protect the persons located in the building, building part or mobile facility, the fresh air is generally filtered in order to reduce the amount of viruses in relation to the outside air. This also includes the air circulating in the building, building part or mobile facility (circulating air). In order to reduce the amount of viruses in the ambient air, the circulating air is generally also filtered. Moreover, to protect the persons outside the building, building part or mobile facility, it may also be expedient to filter the discharged exhaust air. In a preferred embodiment, the planar substrate is used as a filter medium in an air-conditioning system. This includes systems without ventilation function, such as circulation systems and recirculation units and systems with ventilation function, such as ventilation systems and air-conditioning systems. In a further preferred embodiment, the planar substrate is used as a filter medium in a ventilation system of transport means, such as road vehicles, rail vehicles, watercraft or aircraft. The transport means is preferably selected from among passenger cars, buses, trucks, trains, ships and aircraft. Preferred is the use according to the invention of the planar substrate as a filter medium for depleting viruses in the interior spaces of transport means, such as road vehicles, rail vehicles, watercraft or aircraft. The use of the planar substrate as a filter medium for depleting viruses in the passenger compartments of motor vehicles is particularly preferred.

Advantageously, the loaded filter medium also has essentially no more pathogenic material. Used filter materials can thus be disposed of without problems by customary methods, for example thermally.

Viruses may be in the air and other gases in the form of aerosols (airborne particles), wherein the viruses themselves may form the aerosol particles or may be attached to other particulate aerosol components such as dust, water droplets, etc. Filters in ventilation systems are generally in the form of filter arrangements which comprise a plurality of filter components and frequently also have particle-filtering regions in addition to absorbing regions. It is thus possible to effectively clean even complex gas particle systems. The planar substrate according to the invention is advantageously suitable as a filter medium for use in such filter arrangements.

A further subject matter of the present invention is a filter arrangement comprising a filter medium as described above. In a preferred embodiment of the invention, the filter arrangement comprises a particle-filtering region and/or an absorbing region, wherein the filter medium can be comprised by one or both of these regions.

In a particularly preferred embodiment of the invention, the filter arrangement comprises the following components:

A) a particle-filtering region comprising
a particle filter carrier layer, and
a microfiber layer and/or membrane filter layer arranged on the particle filter carrier layer,
optionally a cover layer arranged on the side of the microfiber layer and/or membrane filter layer facing away from the particle filter carrier layer; and/or
B) an absorbent region comprising
an adsorption layer, and
an adsorption carrier layer arranged on the adsorption layer, wherein at least one layer selected from particle filter carrier layer, microfiber layer, membrane filter layer, cover layer, adsorption layer and adsorption carrier layer is composed of a filter medium as described above.

The term “particle filter carrier layer” as used herein is to be understood as meaning a layer which can serve as a carrier layer for a microfiber layer and/or membrane filter layer.

The term “membrane filter layer” as used herein is to be understood as meaning a layer that constitutes a permeable membrane.

The term “cover layer” as used herein is to be understood as meaning a layer which can serve for covering and protecting the microfiber layer and/or membrane filter layer.

The term “adsorption layer” as used herein is to be understood as meaning a layer having an adsorbent. It is preferably selected from the group consisting of activated carbon particles, zeolites, ion exchangers and mixtures thereof. The adsorbent is advantageously arranged statistically randomly in the adsorption layer as a flow-through bulk layer on the adsorption carrier layer.

The term “adsorption carrier layer” as used herein is to be understood as meaning a layer which can serve as a carrier layer for the adsorption layer.

The adsorbing region of the filter arrangement can also consist of a geometrically determined arrangement of the adsorbent, for example as a flow-through honeycomb body of defined cell geometry and/or use of a geometrically defined support structure for mechanically stabilizing an adsorption layer.

It is conceivable for the filter arrangement to comprise only the particle-filtering region or the absorbing region. Advantageously, however, the filter arrangement has both the particle-filtering region and the absorbing region, as this provides a particularly effective filter arrangement. In this case, the two regions are preferably arranged in such a way that the adsorption layer is arranged on the side of the microfiber layer, membrane filter layer or cover layer that faces away from the particle filter carrier layer. In addition, the filter arrangement is preferably arranged in use in such a way that the particle-filtering region is arranged upstream of the absorbing region in relation to the direction of flow. As a result, active substances present in the absorbent region, for example the first and second acid, can be protected from being contaminated with foreign particles present in the incoming air.

According to the invention, at least one layer selected from the particle filter carrier layer, microfiber layer, membrane filter layer, cover layer, adsorption layer and adsorption carrier layer is composed of a filter medium as described above and consequently has the combination of first and second acid according to the invention. The above-described specific embodiments of the filter medium can be transferred to the respective corresponding layers of the filter arrangement. In principle, only a single layer or also different layers of the filter arrangement can have the combination of the first and second acid according to the invention.

Introducing the first and second acid into the particle filter carrier layer is advantageous in that the latter usually faces the air stream as the first layer of the filter arrangement and that allergen-containing particles and dusts of the air stream can thus be deactivated before penetrating the lower layers of the filter arrangement.

In a preferred embodiment of the invention, the first and second acid is contained in the cover layer. This embodiment is advantageous in that the layers upstream in the filter arrangement are not influenced in terms of their filtering properties. Moreover, here too, the first and second acid can be protected from being contaminated with foreign particles present in the incoming air. This arrangement can be even more advantageous if the first and second acid is present neither in the particle filter carrier layer nor in the microfiber layer or the membrane filter layer.

Introducing the first and second acid into the adsorption layer is advantageous in that adsorption layers generally provide high specific surfaces (approx. 1000 m<2>/g when using activated carbon), and, therefore, a large reactive surface is available for allergen deactivation. Moreover, here too, the first and second acid can be protected from being contaminated with foreign particles present in the incoming air by the particle-filtering region or by the adsorption carrier layer.

Introducing the first and second acid into the adsorption carrier layer is advantageous in that the layers upstream in the filter arrangement are not influenced in terms of their filtering properties by the introduction of the first and second acid into the adsorption carrier layer. Moreover, the first and second acid can be protected from being contaminated with foreign particles present in the incoming air by the particle-filtering region.

In a particularly preferred embodiment according to the invention, the filter arrangement has the following structure with respect to the flow direction: Particle filter carrier layer, microfiber layer, adsorption layer and adsorption carrier layer. The particle filter carrier layer is advantageously arranged upstream in use.

As already explained above, the carrier materials for particle filter carrier layer, microfiber layer, membrane filter layer, cover layer and adsorption carrier layer can advantageously be nonwovens, wovens, warp-knitted fabrics and/or papers.

It has also proven suitable to set the amount of the first acid in the filter arrangement to from 0.003 wt % to 30 wt %, preferably from 0.1 wt % to 24 wt %, more preferably from 0.2 wt % to 18 wt %, even more preferably from 0.25 wt % to 15 wt %, and in particular from 0.3 wt % to 12 wt % in each case based on the total weight of the filter arrangement. It has moreover proven suitable to adjust the amount of the second acid in the filter arrangement to from 0.0001 wt % to 10 wt %, more preferably from 0.0003 wt % to 5 wt %, more preferably from 0.0006 wt % to 1 wt %, even more preferably from 0.001 wt % to 0.6 wt % and in particular from 0.003 wt % to 0.12 wt %, in each case based on the total weight of the filter arrangement.

In a preferred embodiment of the invention, the adsorption carrier layer and/or the particle filter carrier layer comprises a nonwoven, preferably selected from spunbond nonwovens, with an average fiber diameter in the range of from 20 to 70 μm, preferably from 20 to 50 μm, in particular from 20 to 50 μm and/or staple fiber nonwovens with an average fiber diameter of from 5 to 60 μm, preferably from 10 to 50 μm, in particular from 10 to 35 μm and/or an average fiber length of from 10 to 100 mm, preferably from 30 to 80 mm. Further advantageously, the microfiber layer and/or membrane filter layer has a nonwoven, preferably selected from meltblown fiber nonwovens, having an average fiber diameter of from 1 μm to 10 μm. Further advantageously, the cover layer comprises a nonwoven, preferably selected from spunbond nonwovens, having an average fiber diameter in the range of from 20 to 60 μm and/or staple fiber nonwovens having an average fiber diameter of 10 to 50 μm.

A particularly preferred embodiment according to the invention comprises the embodiment of the adsorption carrier layer, the particle filter carrier layer, the microfiber layer, the membrane filter layer and/or the cover layer as a nonwoven impregnated and/or coated with the first and second acid, as described above.

The invention is explained with reference to the following non-limiting examples.

### By Way of Example

A carrier nonwoven made of polyester spunbond nonwoven (grammage: 60 g/m²) was antivirally equipped with a mixture of lauric acid and citric acid. The antiviral doping of the carrier nonwoven was carried out by applying a mixture of the active agents in aqueous solution onto the carrier nonwoven and subsequently drying the now finished nonwoven in order to thereby obtain a sample for analysis. The nonwoven thus finished contained, for example, lauric acid in a weight amount of 0.2 mg and citric acid in a weight amount of 10 mg, based in each case on 100 mg of nonwoven.

The size of the samples used in the test was 20 mm×20 mm. The antiviral activity was tested in accordance with ISO 18184:2019-06. Each sample cut in 20 mm×20 mm pieces was soaked in solutions of known starting virus concentration of viral strains H1N1 or HCoV229E at 25° C. After soaking for not more than two hours, the supernatant is pipetted off and the viral concentration in each sample is determined. A viral pathogen reduction factor of at least 3.0 log stages was achieved in each case.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A method, comprising:
using a planar substrate comprising at least one acid-functionalized layer comprising at least one first acid having a pks1 value of 0 to 7 and at least one different second acid or derivative thereof selected from a group consisting of C₈ to C₁₈ fatty acids, esters, amides, and mixtures thereof, as a filter medium for depleting viruses from air and other gases.

2. The method of claim 1, wherein the planar substrate is free of polyoxyethylene(20)-sorbitan-monooleate (polysorbate 80).

3. The method of claim 1, wherein the method is used for depleting viruses from the air of buildings, building parts and mobile facilities.

4. The method of claim 1, wherein the method is used for depleting SARS-associated coronavirus, MERS-CoV, and influenza virus A from the air and other gases.

5. The method of claim 1, wherein the first acid is selected from a group consisting of fruit acids and mixtures thereof.

6. The method of claim 1, wherein the second acid is selected from a group consisting of C₈ to C₁₆ fatty acids and mixtures thereof.

7. The method of claim 1, wherein a weight ratio between the first acid and the second acid in the filter medium is from 10,000:1 to 1:1.

8. The method of claim 1, wherein the acid-functionalized layer comprises the first acid in an amount of from 0.1 wt % to 30 wt % and the second acid is present in an amount of less than 10 wt %, in each case based on a total weight of the acid-functionalized layer.

9. The method of claim 1, wherein the acid-functionalized layer comprises at least one carrier material.

10. The method of claim 1, wherein the acid-functionalized layer comprises an impregnated and/or coated nonwoven.

11. The method of claim 1, wherein the first acid and/or the second acid is introduced as a pourable or free-flowing solid onto and/or into the acid-functionalized layer.

12. The method of claim 1, wherein during production of the acid-functionalized layer, a layer to be functionalized with acid is treated with a surfactant as wetting agent before and/or simultaneously with application of the second acid.

13. The method of claim 1, wherein during production a layer to be functionalized with acid is treated with a fungicidal substance before and/or simultaneously with application of the second acid.

14. The method of claim 1 in a filter arrangement for filtration of gas particle systems, comprising:
a particle-filtering region, comprising:
a particle filter carrier layer, and
a microfiber layer and/or membrane filter layer arranged on the particle filter carrier layer, and/or
an absorbent region, comprising:
an adsorption layer, and
an adsorption carrier layer arranged on the adsorption layer,
wherein at least one layer selected from the particle filter carrier layer, microfiber layer, membrane filter layer, cover layer, adsorption layer, and adsorption carrier layer comprises the filter medium.

15. A method for depleting viruses from air or other gases, comprising:
introducing virus-enriched air or gas into a filter device comprising at least one planar substrate of claim 1 as the filter medium;
directing the air or the gas through the filter medium or contacting the air or the gas with the filter medium to obtain air depleted of viruses or gas depleted of viruses; and
discharging air depleted of viruses or the gas depleted of viruses from the filter device.

16. The method of claim 15, wherein depletion of the viruses in the air or other gases is performed by circulating air circulation.

17. The planar substrate of claim 1, wherein the planar substrate is configured to deplete viral pathogens from air or other gases.

18. The method of claim 3, wherein the method is used for depleting for depleting viruses from incoming air and/or circulating air and/or exhaust air of buildings, building parts and mobile facilities, in particular for depleting viruses from the interior spaces of transport means, such as road vehicles, rail vehicles, watercraft or aircraft.

19. The method of claim 18, wherein the method is used for depleting viruses from interior spaces of transport means comprising road vehicles, rail vehicles, watercraft, or aircraft.

20. The method of claim 4, wherein the method is used for depleting SARS-CoV-2, MERS-CoV, and influenza virus A variant H1N1 from the air and other gases.
